(19) United States
(12) Patent Application Publication
(10) Pub. No.: US 2024/0425519 A1
(43) Pub. Date: Dec. 26, 2024

(54) NOVEL SPIROCYCLIC COMPOUNDS AS KLHDC2 LIGANDS

(71) Applicants: University of Washington, Seattle, WA (US); ACCUTAR BIOTECHNOLOGY INC., Brookyln, NY (US)

(72) Inventors: Ning ZHENG, Seattle, WA (US); Domnita Valeria RUSNAC, Seattle, WA (US); Jie FAN, Seattle, WA (US); Yimin QIAN, Seattle, WA (US); Ke LIU, Seattle, WA (US)

(73) Assignees: University of Washington, Seattle, WA (US); ACCUTAR BIOTECHNOLOGY INC., Brookyln, NY (US)

(21) Appl. No.: 18/707,106

(22) PCT Filed: Oct. 31, 2022
(86) PCT No.: PCT/US2022/048403

§ 371 Date: May 2, 2024

Related U.S. Application Data
(60) Provisional application No. 63/275,667, filed on Nov. 4, 2021.

(51) Int. Cl. C07D 498/10; A61K 31/553; C07D 487/10; C07D 519/00

(52) U.S. Cl. CPC C07D 498/10; A61K 31/553; C07D 487/10; C07D 519/00

(57) ABSTRACT

Spirocyclic compounds that are KLHDC2 ligands and methods for inhibiting the enzymatic activity of KLHDC2 using the ligands.

### CROSS-REFERENCE TO RELATED APPLICATION

This application claims the benefit of U.S. Patent Application No. 63/275,687, filed Nov. 4, 2021, expressly incorporated herein by reference in its entirety.

### BACKGROUND

KLHDC2 is a ubiquitin ligase enzyme that promotes the degradation of various proteins by binding and modifying them. In humans, aberrant proteins can be deleterious to cells and are cleared by the ubiquitin-proteasome system. A group of C-end degrons has recently been identified in some of these abnormal polypeptides, which are recognized by specific cullin-RING ubiquitin E3 ligases (CRLs). Recently, three crystal structures have been reported of a CRL2 substrate receptor, KLHDC2, in complex with the diglycine-ending C-end degrons of two early terminated selenoproteins and the N-terminal proteolytic fragment of USP1. The E3 recognizes the degron peptides in a similarly coiled conformation and cradles their C-terminal diglycine with a deep surface pocket. By hydrogen bonding with multiple backbone carbonyls of the peptides, KLHDC2 further locks in the otherwise degenerate degrons with a compact interface and unexpected high affinities. In a competition assay, a 12-amino acid C-end diglycine degron peptide interacts with KLHDC2 with an affinity in the single digit nanomolar range.

A need exists for compounds that bind to KLHDC2 and that inhibit KLHDC2 enzymatic activity. The present invention seeks to fulfill this need and provides further related advantages.

### SUMMARY

In one aspect, the disclosure provides KLHDC2 ligands.

In certain embodiments, the KLHDC2 ligands described herein have formula (I):

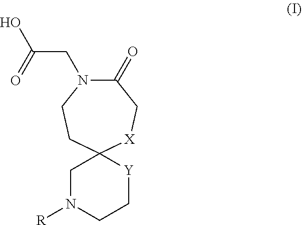

or a pharmaceutically acceptable salt or ester thereof,
wherein
X is CH₂ or O;
Y is NCH₃ or CH₂; and
R is alkylcarbonyl, arylcarbonyl, cycloalkylcarbonyl, alkoxycarbonyl, aryloxycarbonyl, heterocyclic alkylcarbonyl, heteroarylcarbonyl, alkylaminocarbonyl, arylaminocarbonyl, alkyl, aryl, or heteroaryl.

In other embodiments, the KLHDC2 ligands have formula (II):

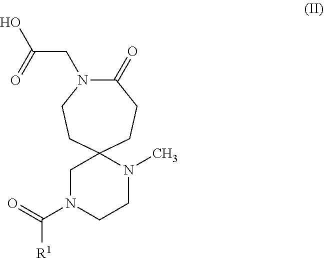

or a pharmaceutically acceptable salt or ester thereof,
wherein R¹ is alkyl, aryl, cycloalkyl, alkoxy, aryloxy, heterocyclic alkyl, heteroaryl, alkylamino, or arylamino.

In certain of these embodiments, R¹ is selected from
(a) C1-C6 alkyl (straight chain or branched) or C3-C6 cycloalkyl, optionally substituted with one or more of a phenyl, halo (e.g., fluoro), hydroxy, phenoxy, C1-C3 alkoxy, or amino (or protected amino);
(b) azetidinyl [(CH₂)₃N—], pyrrolidinyl [(CH₂)₄N—], or piperidinyl [(CH₂)₅N—], wherein the nitrogen is acylated or alkylated;
(c) —N(CH₂)ₙC₆H₅, wherein n=0, 1, 2, 3; or
(d) amino C4-C6 cycloalkyl.

Representative compounds of formula (II) include those shown in Table 1.

In further embodiments, the KLHDC2 ligands have formula (III):

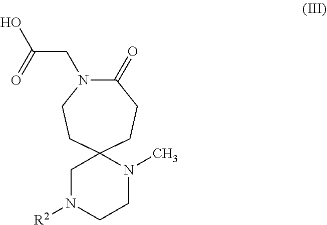

or a pharmaceutically acceptable salt or ester thereof,
wherein R² is alkyl, heteroatom substituted alkyl, cycloalkyl, bicyclic alkyl, aryl, heteroaryl, bicyclic aryl, or heterobicyclic aryl.

In certain of these embodiments, R² is an optionally substituted 2- or 4-pyrimidinyl.

Representative compounds of formula (III) include those shown in Table 3.

In other embodiments, the KLHDC2 ligands have the formula (IV):

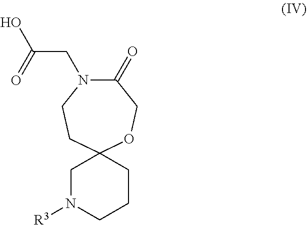

or a pharmaceutically acceptable salt or ester thereof,
wherein R³ is alkylcarbonyl, arylcarbonyl, cycloalkylcarbonyl, alkoxycarbonyl, aryloxycarbonyl, heterocyclic alkylcarbonyl, heteroarylcarbonyl, alkylaminocarbonyl, or arylaminocarbonyl.

In certain of these embodiments, R³ is selected from
(a) —C(═O)C1-C6 alkyl (straight chain or branched) optionally substituted with phenyl;
(b) —C(═O)C3-C6 cycloalkyl; or
(c) optionally substituted 4-pyrimidinyl.

Representative compounds of formula (IV) include those shown in Table 5.

In another aspect, the disclosure provides methods for inhibiting the enzymatic activity of KLHDC2 using the ligands described herein. In certain embodiments, the invention provides a method for inhibiting KLHDC2 enzymatic activity in a subject, comprising contacting KLHDC2 with an amount of a compound of formulae (I)-(IV), or a pharmaceutically acceptable salt or ester thereof, effective to inhibit KLHDC2 enzymatic activity by inhibiting the KLHDC2-protein substrate binding interaction.

### DETAILED DESCRIPTION

KLHDC2 is a ubiquitin ligase enzyme that promotes the degradation of various proteins by binding and modifying them. The compounds described herein bind to the same site on KLHDC2 where its substrate proteins bind. In doing so, these compounds are KLHDC2 ligands that inhibit the interaction between the ubiquitin ligase and its substrate proteins and block the enzymatic activity of KLHDC2.

In one aspect, the invention provides KLHDC2 ligands that bind to KLHDC2 with high affinity and inhibit the enzymatic activity of KLHDC2.

In another aspect, the invention provides methods for inhibiting KLHDC2 enzymatic activity (via inhibiting the KLHDC2-protein substrate binding interaction) using the ligands.

### KLHDC2 Ligands

In one aspect, the invention provides KLHDC2 ligands (i.e., compounds that bind to KLHDC2). These compounds bind to the deep degron-binding pocket of KLHDC2.

The KLHDC2 ligands described herein have formula (I):

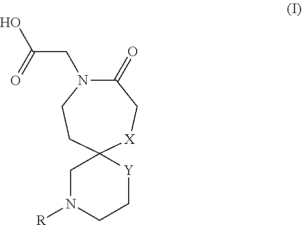

or a pharmaceutically acceptable salt or ester thereof,
wherein
X is CH₂ or O;
Y is NCH₃ or CH₂; and
R is alkylcarbonyl, arylcarbonyl, cycloalkylcarbonyl, alkoxycarbonyl, aryloxycarbonyl, heterocyclic alkylcarbonyl, heteroarylcarbonyl, alkylaminocarbonyl, arylaminocarbonyl, alkyl, aryl, or heteroaryl.

In certain embodiments, the KLHDC2 ligands have formula (II):

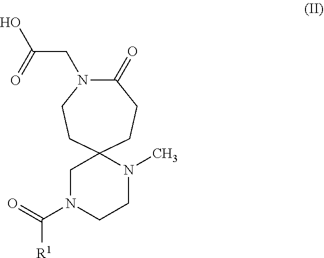

or a pharmaceutically acceptable salt or ester thereof,
wherein R¹ is alkyl, aryl, cycloalkyl, alkoxy, aryloxy, heterocyclic alkyl, heteroaryl, alkylamino, or arylamino.

In certain of these embodiments, R¹ is selected from
(a) C1-C6 alkyl (straight chain or branched) or C3-C6 cycloalkyl, optionally substituted with one or more of a phenyl, halo (e.g., fluoro), hydroxy, phenoxy, C1-C3 alkoxy, or amino (or protected amino [e.g., —NH—(C═O)OtBu]);
(b) azetidinyl [(CH₂)₃N—], pyrrolidinyl [(CH₂)₄N—], or piperidinyl [(CH₂)₅N—], wherein the nitrogen is acylated [e.g., —(C═O)CH₃, —(C═O)C6H₅, or —(C═O)OtBu] or alkylated [e.g., —CH₂C₆H₅ or —CH₂C₅H₄N];
(c) —N(CH₂)ₙC₆H₅, wherein n=0, 1, 2, 3; or
(d) amino C4-C6 cycloalkyl [—NH—(CH₂)ₙ, n=3-6].

Representative compounds of formula (II) include those shown in Table 1.

TABLE 1
Representative compounds of formula (II).

| Compound | Chemical Structure | IUPAC Name |
|---|---|---|
| AC1032 | 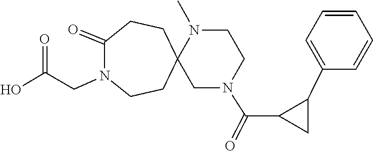 [SMILES: CN1CCN(C(=O)C2CC2c2ccccc2)CC12CCC(=O)N(CC(=O)O)CC2] | 2-(1-methyl-10-oxo-4-(2- phenylcyclopropane-1- carbonyl)-1,4,9-triaza- spiro[5.6]dodecan-9-yl)- acetic acid |
| AC1033 | 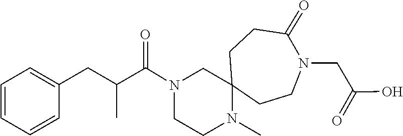 [SMILES: CC(Cc1ccccc1)C(=O)N1CCN(C)C2(CCC(=O)N(CC(=O)O)CC2)C1] | 2-(1-methyl-4-(2-methyl- 3-phenylpropanoyl)-10- oxo-1,4,9-triazaspiro[5.6]- dodecan-9-yl)acetic acid |
| AC1034 | 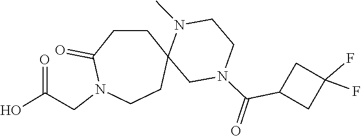 [SMILES: CN1CCN(C(=O)C2CC(F)(F)C2)CC12CCC(=O)N(CC(=O)O)CC2] | 2-(4-(3,3-difluorocyclo- butane-1-carbonyl)-1- methyl-10-oxo-1,4,9- triazaspiro[5.6]dodecan- 9-yl)acetic acid |
| AC1045 | 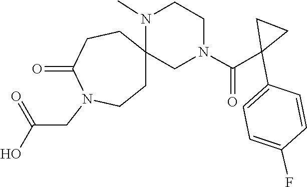 [SMILES: CN1CCN(C(=O)C2(c3ccc(F)cc3)CC2)CC12CCC(=O)N(CC(=O)O)CC2] | 2-(4-(1-(4-fluorophenyl)- cyclopropane-1-carbon- yl)-1-methyl-10-oxo- 1,4,9-triazaspiro[5.6]- dodecan-9-yl)acetic acid |
| AC1046 | 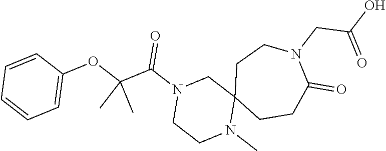 [SMILES: CN1CCN(C(=O)C(C)(C)Oc2ccccc2)CC12CCC(=O)N(CC(=O)O)CC2] | 2-(1-methyl-4-(2-methyl- 2-phenoxypropanoyl)-10- oxo-1,4,9-triazaspiro[5.6]- dodecan-9-yl)acetic acid |
| AC1047 | 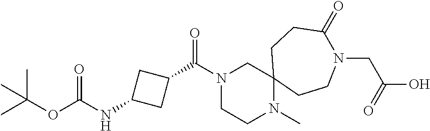 [SMILES: CN1CCN(C(=O)[C@H]2C[C@@H](NC(=O)OC(C)(C)C)C2)CC12CCC(=O)N(CC(=O)O)CC2] | 2-(4-((1,3-trans)-3-((tert- butoxycarbonyl)amino)- cyclobutane-1-carbonyl)- 1-methyl-10-oxo-1,4,9- triazaspiro[5.6]dodecan- 9-yl)acetic acid |
| AC1048 | 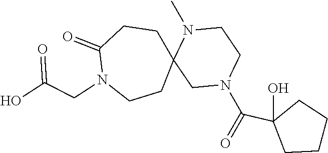 [SMILES: CN1CCN(C(=O)C2(O)CCCC2)CC12CCC(=O)N(CC(=O)O)CC2] | 2-(4-(1-hydroxycyclopen- tane-1-carbonyl)-1-meth- yl-10-oxo-1,4,9-triaza- spiro[5.6]dodecan-9-yl)- acetic acid |
| AC1049 | 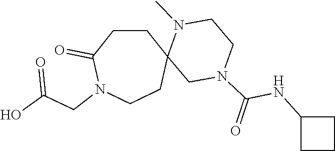 [SMILES: CN1CCN(C(=O)NC2CCC2)CC12CCC(=O)N(CC(=O)O)CC2] | 2-(4-(cyclobutylcarbam- oyl)-1-methyl-10-oxo- 1,4,9-triazaspiro[5.6]- dodecan-9-yl)acetic acid |
| AC1050 | 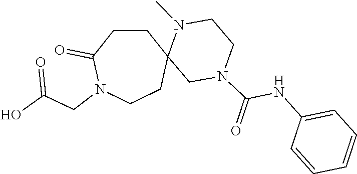 [SMILES: CN1CCN(C(=O)Nc2ccccc2)CC12CCC(=O)N(CC(=O)O)CC2] | 2-(1-methyl-10-oxo-4- (phenylcarbamoyl)-1,4,9- triazaspiro[5.6]dodecan- 9-yl)acetic acid |
| AC1051 | 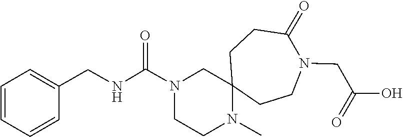 [SMILES: CN1CCN(C(=O)NCc2ccccc2)CC12CCC(=O)N(CC(=O)O)CC2] | 2-(4-(benzylcarbamoyl)- 1-methyl-10-oxo-1,4,9- triazaspiro[5.6]dodecan- 9-yl)acetic acid |
| AC1052 | 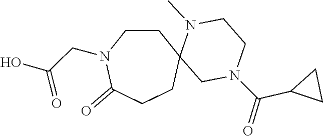 [SMILES: CN1CCN(C(=O)C2CC2)CC12CCC(=O)N(CC(=O)O)CC2] | 2-(4-(cyclopropanecar- bonyl)-1-methyl-10-oxo- 1,4,9-triazaspiro[5.6]- dodecan-9-yl)acetic acid |
| AC1062 | 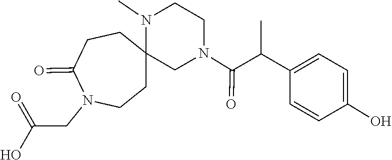 [SMILES: CC(C(=O)N1CCN(C)C2(CCC(=O)N(CC(=O)O)CC2)C1)c1ccc(O)cc1] | 2-(4-(2-(4-hydroxyphen- yl)propanoyl)-1-methyl- 10-oxo-1,4,9-triazaspiro- [5.6]dodecan-9-yl)acetic acid |
| AC1063 | 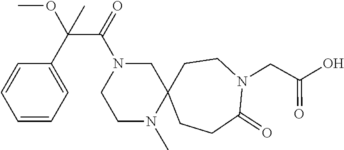 [SMILES: COC(C)(C(=O)N1CCN(C)C2(CCC(=O)N(CC(=O)O)CC2)C1)c1ccccc1] | 2-(4-(2-methoxy-2-phen- ylpropanoyl)-1-methyl- 10-oxo-1,4,9-triazaspiro- [5.6]dodecan-9-yl)acetic acid |
| AC1064 | 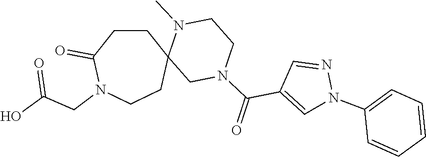 [SMILES: CN1CCN(C(=O)c2cnn(-c3ccccc3)c2)CC12CCC(=O)N(CC(=O)O)CC2] | 2-(1-methyl-10-oxo-4-(1- phenyl-1H-pyrazole-4- carbonyl)-1,4,9-triaza- spiro[5.6]dodecan-9-yl)- acetic acid |
| AC1065 | 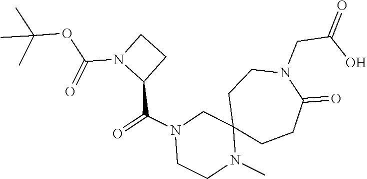 [SMILES: CN1CCN(C(=O)[C@@H]2CCN2C(=O)OC(C)(C)C)CC12CCC(=O)N(CC(=O)O)CC2] | 2-(4-((S)-1-(tert-butoxy- carbonyl)azetidine-2- carbonyl)-1-methyl-10- oxo-1,4,9-triazaspiro[5.6]- dodecan-9-yl)acetic acid |
| AC1066 | 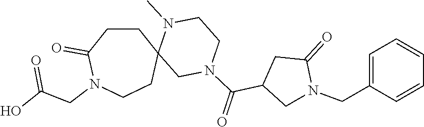 [SMILES: CN1CCN(C(=O)C2CC(=O)N(Cc3ccccc3)C2)CC12CCC(=O)N(CC(=O)O)CC2] | 2-(4-(1-benzyl-5-oxo- pyrrolidine-3-carbonyl)- 1-methyl-10-oxo-1,4,9- triazaspiro[5.6]dodecan- 9-yl)acetic acid |
| AC1068 | 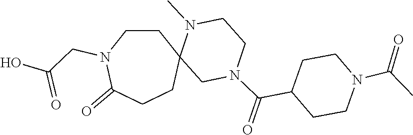 [SMILES: CC(=O)N1CCC(C(=O)N2CCN(C)C3(CCC(=O)N(CC(=O)O)CC3)C2)CC1] | 2-(4-(1-acetylpiperidine- 4-carbonyl)-1-methyl-10- oxo-1,4,9-triazaspiro- [5.6]dodecan-9-yl)acetic acid |
| AC1069 | 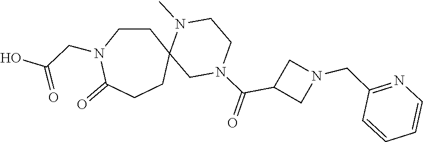 [SMILES: CN1CCN(C(=O)C2CN(Cc3ccccn3)C2)CC12CCC(=O)N(CC(=O)O)CC2] | 2-(1-methyl-10-oxo-4-(1- (pyridin-2-ylmethyl)azeti- dine-3-carbonyl)-1,4,9- triazaspiro[5.6]dodecan- 9-yl)acetic acid |
| AC1070 | 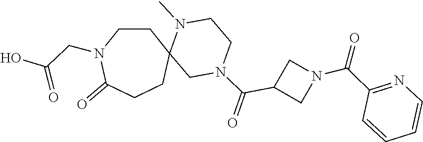 [SMILES: CN1CCN(C(=O)C2CN(C(=O)c3ccccn3)C2)CC12CCC(=O)N(CC(=O)O)CC2] | 2-(4-(1-benzoylazetidine- 3-carbonyl)-1-methyl-10- oxo-1,4,9-triazaspiro- [5.6]dodecan-9-yl)acetic acid |
| AC1071 | 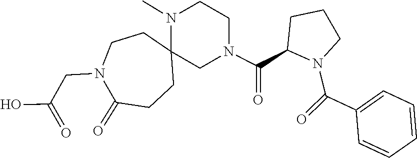 [SMILES: CN1CCN(C(=O)[C@H]2CCCN2C(=O)c2ccccc2)CC12CCC(=O)N(CC(=O)O)CC2] | 2-(4-(benzoyl-D-prolyl)- 1-methyl-10-oxo-1,4,9- triazaspiro[5.6]dodecan- 9-yl)acetic acid |
| AC1072 | 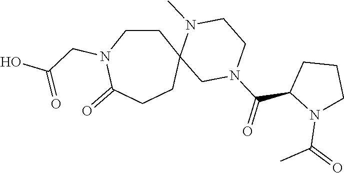 [SMILES: CC(=O)N1CCC[C@@H]1C(=O)N1CCN(C)C2(CCC(=O)N(CC(=O)O)CC2)C1] | 2-(4-(acetyl-D-prolyl)-1- methyl-10-oxo-1,4,9- triazaspiro[5.6]dodecan- 9-yl)acetic acid |

The KLHDC2 binding activities (IC₅₀, μM) of representative compounds are compared in Table 2.

TABLE 2
KLHDC2 binding activities (IC₅₀, μM)
of representative formula (II) compounds.

| Compound | IC₅₀ (μM) |
|---|---|
| AC1032 | 32.04 |
| AC1033 | 56.35 |
| AC1034 | 294.7 |
| AC1045 | 347.8 |
| AC1046 | 202.3 |
| AC1047 | 493.4 |
| AC1048 | 116.1 |
| AC1049 | 91.68 |
| AC1050 | 33.63 |
| AC1051 | 45.89 |
| AC1052 | 100.1 |
| AC1062 | 19.76 |
| AC1063 | 130.9 |
| AC1064 | 147.7 |
| AC1065 | 496.2 |
| AC1066 | 65.89 |
| AC1068 | 191.3 |
| AC1069 | 4.603 |
| AC1070 | 198.5 |
| AC1071 | 45.39 |
| AC1072 | 12.47 |

In other embodiments, the KLHDC2 ligands have formula (III):

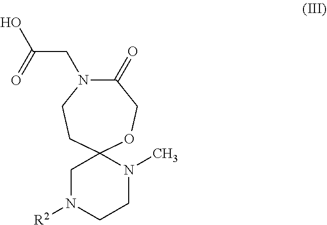

or a pharmaceutically acceptable salt or ester thereof,
wherein R² is alkyl, heteroatom substituted alkyl, cycloalkyl, bicyclic alkyl, aryl, heteroaryl, bicyclic aryl, or heterobicyclic aryl.

In certain of these embodiments, R² is an optionally substituted 2- or 4-pyrimidinyl.

Representative compounds of formula (III) include those shown in Table 3.

TABLE 3
Representative compounds of formula (III).

| Compound | Chemical Structure | IUPAC Name |
|---|---|---|
| AC1030 | 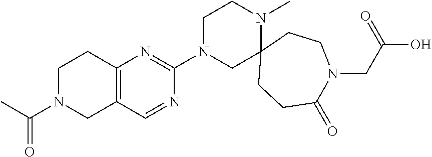 [SMILES: CC(=O)N1CCc2nc(N3CCN(C)C4(CCC(=O)N(CC(=O)O)CC4)C3)ncc2C1] | 2-(4-(6-acetyl-5,6,7,8-tetra- hydropyrido[4,3-d]pyrimidin- 2-yl)-1-methyl-10-oxo-1,4,9- triazaspiro[5.6]dodecan-9-yl)- acetic acid |
| AC1031 | 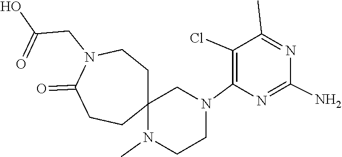 [SMILES: Cc1nc(N)nc(N2CCN(C)C3(CCC(=O)N(CC(=O)O)CC3)C2)c1Cl] | 2-(4-(2-amino-5-chloro-6- methylpyrimidin-4-yl)-1- methyl-10-oxo-1,4,9-triaza- spiro[5.6]dodecan-9-yl)acetic acid |
| AC1035 | 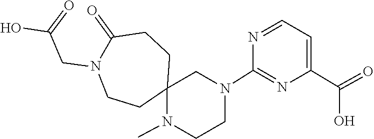 [SMILES: CN1CCN(c2nccc(C(=O)O)n2)CC12CCC(=O)N(CC(=O)O)CC2] | 2-(9-(carboxymethyl)-1-meth- yl-10-oxo-1,4,9-triazaspiro- [5.6]dodecan-4-yl)pyrimidine- 4-carboxylic acid |

The KLHDC2 binding activities (IC₅₀, μM) of representative formula (III) compounds are summarized in Table 4.

TABLE 4
KLHDC2 binding activities (IC₅₀, μM)
of representative formula (III) compounds.

| Compound | IC₅₀ (μM) |
|---|---|
| AC1030 | 444.9 |
| AC1031 | 388.9 |
| AC1035 | 123.1 |

In further embodiments, the KLHDC2 ligands have the formula (IV):

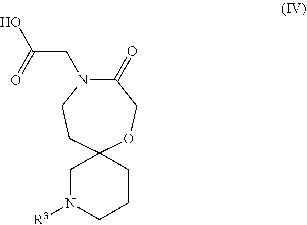

or a pharmaceutically acceptable salt or ester thereof,
wherein R³ is alkylcarbonyl, arylcarbonyl, cycloalkylcarbonyl, alkoxycarbonyl, aryloxycarbonyl, heterocyclic alkylcarbonyl, heteroarylcarbonyl, alkylaminocarbonyl, or arylaminocarbonyl.

In certain of these embodiments, R³ is selected from
(a) —C(═O)C1-C6 alkyl (straight chain or branched) optionally substituted with phenyl;
(b) —C(═O)C3-C6 cycloalkyl; or
(c) optionally substituted 4-pyrimidinyl.

Representative compounds of formula (IV) include those shown in Table 5.

TABLE 5
Representative compounds of formula (IV).

| Compound | Chemical Structure | IUPAC Name |
|---|---|---|
| AC890 | 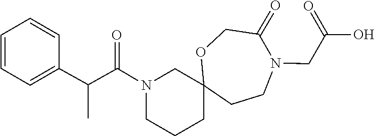 [SMILES: CC(C(=O)N1CCCC2(CCN(CC(=O)O)C(=O)CO2)C1)c1ccccc1] | 2-(9-oxo-2-(2-phenylpropano- yl)-7-oxa-2,10-diazaspiro[5.6]-dodecan-10-yl)acetic acid |
| AC978 | 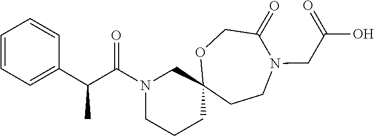 [SMILES: C[C@H](C(=O)N1CCC[C@]2(CCN(CC(=O)O)C(=O)CO2)C1)c1ccccc1] | 2-((S)-9-oxo-2-((S)-2-phenyl- propanoyl)-7-oxa-2,10-diaza- spiro[5.6]dodecan-10-yl)acetic acid |
| AC979 | 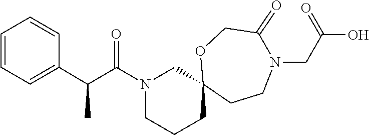 [SMILES: C[C@H](C(=O)N1CCC[C@@]2(CCN(CC(=O)O)C(=O)CO2)C1)c1ccccc1] | 2-((R)-9-oxo-2-((S)-2-phenyl- propanoyl)-7-oxa-2,10-diaza- spiro[5.6]dodecan-10-yl)acetic acid |
| AC1028 | 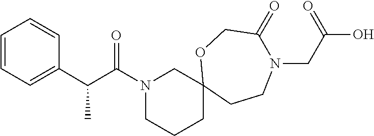 [SMILES: C[C@@H](C(=O)N1CCCC2(CCN(CC(=O)O)C(=O)CO2)C1)c1ccccc1] | 2-(9-oxo-2-((R)-2-phenylpro- panoyl)-7-oxa-2,10-diaza- spiro[5.6]dodecan-10-yl)acetic acid |
| AC903 | 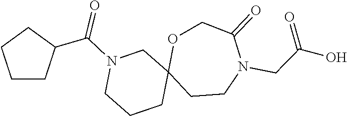 [SMILES: O=C(O)CN1CCC2(CCCN(C(=O)C3CCCC3)C2)OCC1=O] | 2-(2-(cyclopentanecarbonyl)- 9-oxo-7-oxa-2,10-diazaspiro- [5.6]dodecan-10-yl)acetic acid |
| AC904 | 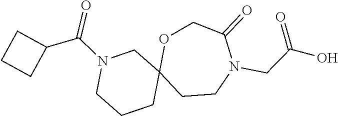 [SMILES: O=C(O)CN1CCC2(CCCN(C(=O)C3CCC3)C2)OCC1=O] | 2-(2-(cyclobutanecarbonyl)-9- oxo-7-oxa-2,10-diazaspiro- [5.6]dodecan-10-yl)acetic acid |
| AC935 | 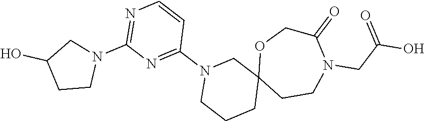 [SMILES: O=C(O)CN1CCC2(CCCN(c3ccnc(N4CCC(O)C4)n3)C2)OCC1=O] | 2-(2-(2-(3-hydroxypyrrolidin- 1-yl)pyrimidin-4-yl)-9-oxo-7- oxa-2,10-diazaspiro[5.6]- dodecan-10-yl)acetic acid |
| AC947 | 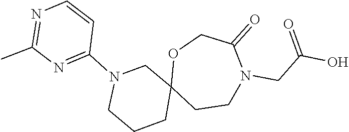 [SMILES: Cc1nccc(N2CCCC3(CCN(CC(=O)O)C(=O)CO3)C2)n1] | 2-(2-(2-methylpyrimidin-4-yl)- 9-oxo-7-oxa-2,10-diazaspiro- [5.6]dodecan-10-yl)acetic acid |
| AC1005 | 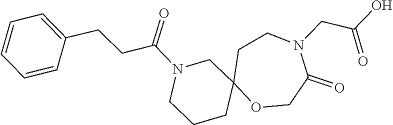 [SMILES: O=C(O)CN1CCC2(CCCN(C(=O)CCc3ccccc3)C2)OCC1=O] | 2-(9-oxo-2-(3-phenylpropan- oyl)-7-oxa-2,10-diazaspiro- [5.6]dodecan-10-yl)acetic acid |

The KLHDC2 binding activities (IC₅₀, μM) of representative compounds are summarized in Table 6.

TABLE 6
KLHDC2 binding activities (IC₅₀, μM)
of representative formula (IV) compounds.

| Compound | IC₅₀ (μM) |
|---|---|
| AC890 | 50 |
| AC0978 | 22.1 |
| AC0979 | 63.7 |
| AC1028 | 39.6 |
| AC903 | 40.1 |
| AC904 | 51 |
| AC935 | 47.5 |
| AC1005 | 8.2 |
| AC947 | 1391 |

In AC890 the benzylic carbon is an (R)/(S) mixture and in AC1028 the benzylic carbon is the (R)-configuration.

The preparations of representative compounds described herein are described in Examples 1-11.

### KLHDC2 Enzymatic Activity Inhibition

In another aspect, the invention provides methods for inhibiting the enzymatic activity of KLHDC2 using the ligands described herein. These ligands inhibit the interaction between the ubiquitin ligase and its substrate proteins and block the enzymatic activity of KLHDC2 (via inhibiting the KLHDC2-protein substrate binding interaction).

In certain embodiments, the invention provides a method for inhibiting KLHDC2 enzymatic activity in a subject, comprising contacting KLHDC2 with an amount of a compound of formulae (I)-(IV), or a pharmaceutically acceptable salt or ester thereof, effective to inhibit KLHDC2 enzymatic activity by inhibiting the KLHDC2-protein substrate binding interaction.

An assay for evaluating ligand binding to KLHDC2 is described in Example 12.

The following examples are provided for the purpose of illustrating, not limiting the invention.

### EXAMPLES

A general synthetic scheme for the preparation of the key intermediate 1-8 is shown in Scheme 1 below.

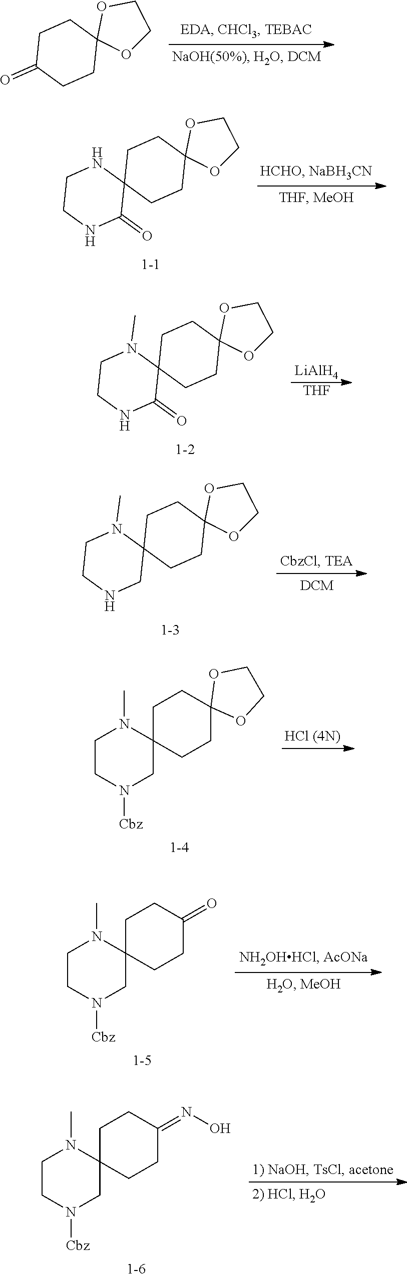

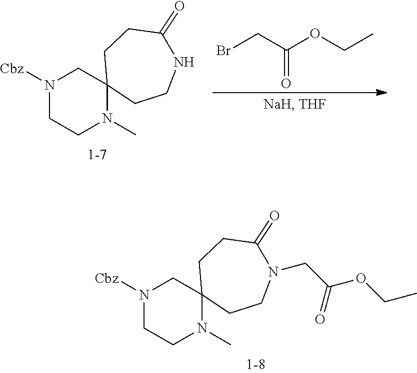

As shown in Scheme 1, the required key intermediate 1-8 can be prepared in 8 steps. The commercial available 1,4-dioxaspiro[4.5]decan-8-one can react with ethylene diamine in chloroform under basic conditions in the presence of phase transfer reagents benzyltriethylammonium chloride to form the spiro lactam 1-1. The amino group in 1-1 can be reacted with formaldehyde under the reductive amination condition to form the N-methyl intermediate 1-2. Reduction of the lactam in 1-2 with lithium aluminum hydride can form the spiro piperazine intermediate 1-3, which can be protected to give the Cbz-protected spiro piperazine intermediate 1-4. Deprotection of the ketal 1-4 under acidic condition can form the corresponding ketone 1-5. The reaction of ketone 1-5 with hydroxylamine can form an oxime 1-6, which can undergo Beckmann rearrangement to form the spiro lactam 1-7. The alkylation of lactam 1-7 with 2-bromoacetic acid ethyl ester will result in the key intermediate 1-8.

The general synthetic scheme for the preparation of representative compounds of the invention is shown in Scheme 2 below.

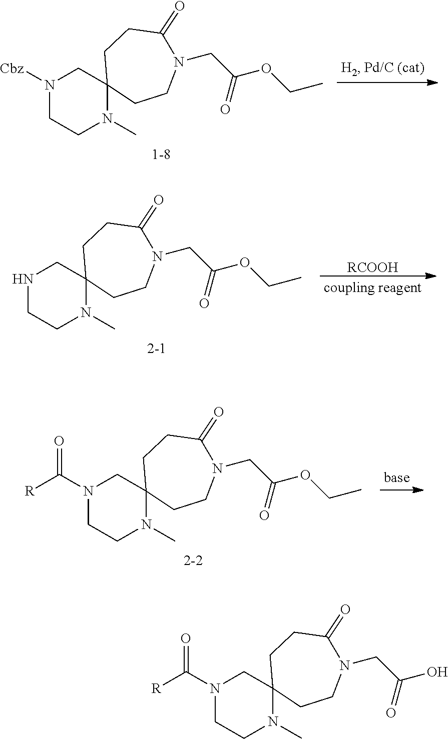

As shown in Scheme 2, hydrogenation in the presence of palladium catalyst will deprotect the Cbz group to form 2-1, which can be coupled with a carboxylic acid using amide coupling reagents to form 2-2. The deprotection of the ester group in 2-2 can be accomplished under basic condition to form the desired carboxylic acid.

The general synthetic scheme for the preparation of key intermediate 3-4 is shown in Scheme 3 below.

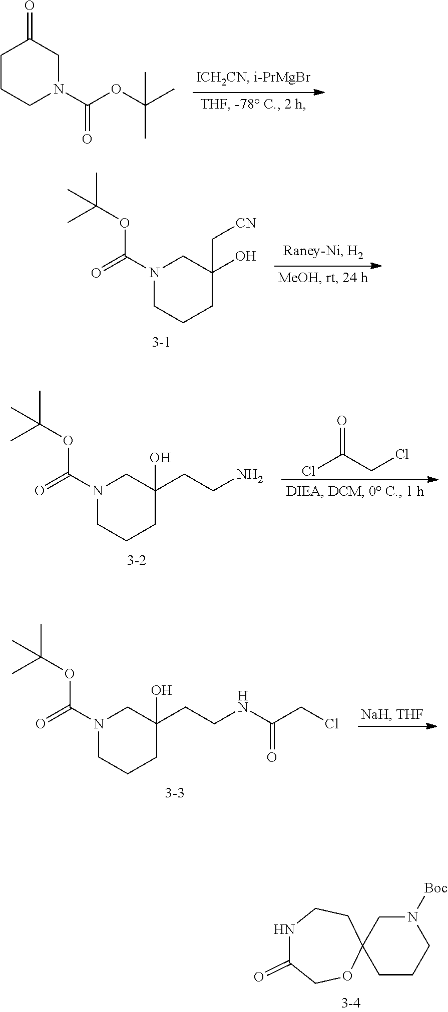

As shown in Scheme 3, reaction of tert-butyl 3-oxopiperidine-1-carboxylate with a Grignard reagent generated in situ can provide the ketone addition product 3-1. The cyano group in 3-1 can be reduced under hydrogenation using Raney Nickel in methanol to generate the desired amine 3-2. Acylation of the amine with 2-chloroacetyl chloride can afford the 3-3 which can be cyclized under basic condition such as sodium hydride to generate 7-oxa-2,10-diazaspiro[5.6]dodecan-9-one, the key intermediate for the synthesis of claimed compounds.

### Preparation of benzyl 9-(2-ethoxy-2-oxoethyl)-1-methyl-10-oxo-1,4,9-triazaspiro[5.6]dodecane-4-carboxylate (Intermediate 1-8)

### Step 1: Synthesis of 1,4-dioxa-9,12-diazadispiro[4.2.5⁸.2⁵]pentadecan-13-one

Sodium hydroxide (564 g, 14.1 mol) in water (564 g) was added dropwise to the solution of Ethylenediamine (EDA) (178 g, 2.96 mol) and benzyltriethylammonium chloride (TEBAC) (32.1 g, 0.141 mol) in DCM (1.2 L) at 5-10° C. After 1,4-dioxaspiro[4.5]decan-8-one (440 g, 2.82 mol) and trichloromethane (436 g, 3.67 mol) were added dropwise at 5-15° C. Then DCM (0.2 L) was added. The reaction was stirred at room temperature for 10 h. The reaction was quenched with ice water (5 L) and extracted with DCM (5 L for three times), The combined organic layer was evaporated in reduced pressure and the residue was purified by recrystallization with isopropanol to give the product as a white solid (300 g, yield 47.06%). LC-MS: m/z 227[M+H]⁺.

### Step 2: Synthesis of 9-methyl-1,4-dioxa-9,12-diazadispiro[4.2.5⁸.2⁵]pentadecan-13-one

To a mixture of 1,4-dioxa-9,12-diazadispiro[4.2.5⁸.2⁵]pentadecan-13-one (300 g, 1.33 mol) in THF (1.5 L) and MeOH (1.5 L) was added formaldehyde (647 g, 7.98 mol) at room temperature. After the mixture was stirred at room temperature for 3 h. sodium cyanoborohydride (134 g, 2.13 mol) was added pointwise at 5-15° C. After the mixture was stirred at room temperature for 30 min. The mixture was quenched with water, adjusted PH to 6-7 with glacial acetic acid, extracted with DCM, evaporated and purified by chromatography on silica-gel (Methanol/DCM=1/25) to give the product as a white solid (247 g, yield 77.5%). LC-MS: m/z 241[M+H]⁺.

### Step 3: Synthesis of 9-methyl-1,4-dioxa-9,12-diazadispiro[4.2.5⁸.2⁵]pentadecane

Lithium aluminum hydride (35.8 g, 0.942 mol) was dissolved in THF (300 mL) as suspension. 9-methyl-1,4-dioxa-9,12-diazadispiro[4.2.5⁸.2⁵]pentadecan-13-one (113 g, 0.471 mol) in THF (1.0 L) was added dropwise at 0-20° C. over 30 min. The mixture was stirred at 65° C. for 2 h. The mixture was quenched by addition of water (35 ml), aqueous sodium hydroxide (30%, 135 ml) at 0° C. The resulting mixture was stirred for 10 mins at 0° C., the solid was removed by a filtration. The filtrate was evaporated to give the product as a colorless oil (100 g, yield 93.9%). LC-MS: m/z 227[M+H]⁺.

### Step 4: Synthesis of benzyl 9-methyl-1,4-dioxa-9,12-diazadispiro[4.2.5⁸.2⁵]pentadecane-12-carboxylate

To a stirred solution of 9-methyl-1,4-dioxa-9,12-diazadispiro[4.2.5⁸.2⁵]pentadecane (100 g, 0.442 mol) in DCM (500 mL) was added TEA (89.3 g, 0.884 mol). To above, Benzyl chloroformate (90.4 g, 0.530 mol) was added dropwise at 0-10° C. under nitrogen atmosphere. The reaction mixture was stirred at room temperature for 2 h. The mixture was quenched with ice water (500 mL), extracted with DCM (500 mL for twice). The combined organic layers were dried over anhydrous sodium sulfate and evaporated under reduced pressure. The residue was purified by chromatography on silica-gel (Methanol/DCM=1/50) to give the product as a white solid (120 g, yield 75.3%). LC-MS: m/z 361[M+H]⁺.

### Step 5: Synthesis of benzyl 1-methyl-9-oxo-1,4-diazaspiro[5.5]undecane-4-carboxylate

Benzyl 9-methyl-1,4-dioxa-9,12-diazadispiro[4.2.5⁸.2⁵]pentadecane-12-carboxylate (300 g, 0.833 mol) in aqueous hydrochloride (4M, 2.0 L) was stirred for 10 h at room temperature. The mixture was adjusted pH to 8-9 with aqueous sat. sodium bicarbonate and extracted with ethyl acetate (2 L for twice). The combined organic layers were dried over anhydrous sodium sulfate and evaporated in reduced pressure to give the product as a yellow oil (234 g, yield 88.86%). LC-MS: m/z 317[M+H]⁺.

### Step 6: Synthesis of benzyl 9-(hydroxyimino)-1-methyl-1,4-diazaspiro[5.5]undecane-4-carboxylate

To a mixture of benzyl 1-methyl-9-oxo-1,4-diazaspiro[5.5]undecane-4-carboxylate (234 g, 0.740 mol) in Methanol (1.0 L) and water (1.0 L) was added sodium acetate (60.7 g, 0.740 mol) and hydroxylamine hydrochloride salt (51.1 g, 0.740 mol) at 0° C. The mixture was stirred at 85° C. for 10 h. The solvent was removed in reduced pressure. The residue was diluted with DCM (500 mL) and washed with brine (100 ml), The organic layer was dried over anhydrous sodium sulfate and evaporated in reduced pressure to give the product as a yellow oil (270 g, crude). LC-MS: m/z 332[M+H]⁺.

### Step 7: Synthesis of benzyl 1-methyl-10-oxo-1,4,9-triazaspiro[5.6]dodecane-4-carboxylate

To a mixture of benzyl 9-(hydroxyimino)-1-methyl-1,4-diazaspiro[5.5]undecane-4-carboxylate (147 g, 0.444 mol) in acetone (1.0 L) was added a solution of sodium hydroxide (151 g, 3.77 mol) in water (800 mL). The mixture was stirred at room temperature for 10 min before tosyl chloride (93.0 g, 0.488 mol) was added. The mixture was stirred for 2 h at room temperature. The PH of resulting mixture was adjusted to 1-2 by dropwise addition of aqueous con. hydrochloride at 0°. The mixture was stirred for 10 h at room temperature. The resulting mixture was adjusted pH to 9-10 with sodium bicarbonate solution and extracted with DCM (500 mL for twice). The combined organic layers were dried with anhydrous sodium sulfate and evaporated under reduced pressure. The residue was purified by chromatography on silica-gel (Methanol/DCM=1/50) to give the product as an off white solid (62.0 g, yield 42.1%). LC-MS: m/z 332[M+H]⁺.

### Step 8: Synthesis of benzyl 9-(2-ethoxy-2-oxoethyl)-1-methyl-10-oxo-1,4,9-triazaspiro[5.6]dodecane-4-carboxylate

To a mixture of benzyl 1-methyl-10-oxo-1,4,9-triazaspiro[5.6]dodecane-4-carboxylate (56.5 g, 0.171 mol) in THF (1.0 L) was added sodium hydride (60% in oil, 10.2 g, 0.256 mol) batchwise at 0° C. The mixture was stirred for 2 h at room temperature before ethyl 2-bromoacetate (34.2 g, 0.205 mol) was added. After stirred for 1 h at room temperature, the resulting suspension was refluxed for 10 h. The mixture was quenched with aqueous ammonium chloride (10%), extracted with DCM (500 mL for twice). The combined organic layers were dried over anhydrous sodium sulfate and evaporated in reduced pressure, The residue was purified by chromatography on silica-gel (Methanol/DCM=1/70) to give the product as a yellow oil (27.53 g, yield 38.7%). LC-MS: m/z 418[M+H]⁺; ¹H NMR (400 MHz, DMSO-d₆) δ 7.30-7.39 (m, 5H), 5.08 (s, 2H), 4.06-4.11 (m, 4H), 3.24-3.44 (m, 6H), 2.59 (br, 2H), 2.33-2.42 (m, 2H), 2.22 (s, 3H), 1.75-1.93 (m, 2H), 1.37-1.43 (m, 2H), 1.17-1.20 (m, 3H).

### Example 1

### Preparation of 2-(4-(acetyl-D-prolyl)-1-methyl-10-oxo-1,4,9-triazaspiro[5.6]dodecan-9-yl) acetic acid (AC1072)

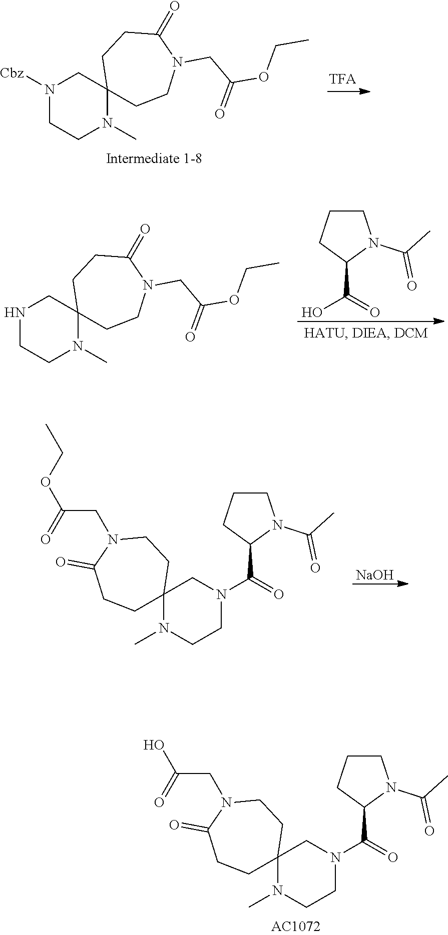

### Step 1: Preparation of ethyl 2-(1-methyl-10-oxo-1,4,9-triazaspiro[5.6]dodecan-9-yl)acetate

The solution of benzyl 9-(2-ethoxy-2-oxoethyl)-1-methyl-10-oxo-1,4,9-triazaspiro[5.6]dode cane-4-carboxylate (130 mg, 0.31 mmol) in TFA (5 mL) was stirred under refluxing for 4 hours. The solvent was removed in vacuum to give the title compound (170 mg, TFA salt, crude) as a brown solid. LC/MS: 284.3 [M+H]⁺.

### Step 2: Preparation of ethyl 2-(4-(acetyl-D-prolyl)-1-methyl-10-oxo-1,4,9-triazaspiro [5.6]dodecan-9-yl) acetate

To a solution containing (2R)-1-acetylpyrrolidine-2-carboxylic acid (16.6 mg, 0.11 mmol), DIEA (27 mg, 0.21 mmol), ethyl 2-{1-methyl-10-oxo-1,4,9-triazaspiro[5.6]dodecan-9-yl}acetate (30 mg, 0.11 mmol) in DCM (5 mL) was added HATU (33 mg, 0.116 mmol). The mixture was stirred at 25° C. for 16 hr. The mixture was diluted with water (10 mL) and extracted with DCM (10 mL×3). The combined organic layers were washed with brine (10 mL) and dried over Na₂SO₄. The organic solvent was evaporated under vacuum to give the titled product (50 mg) as brown oil which was used in the next step without further purification. LC/MS: 423.3 [M+H]⁺.

### Step 3: Preparation of 2-(4-(acetyl-D-prolyl)-1-methyl-10-oxo-1,4,9-triazaspiro[5.6]dodecan-9-yl)acetic acid (AC1072)

To a solution of ethyl 2-(4-{[(2R)-1-acetylpyrrolidin-2-yl]carbonyl}-1-methyl-10-oxo-1,4,9-triazaspiro[5.6]dodecan-9-yl)acetate (40 mg, 0.095 mmol) in THF (4 mL) and H₂O (1 mL) was added 1N NaOH (0.5 mL). The mixture was stirred at 25° C. for 2 hr. The reaction mixture was concentrated in vacuo and the residue was adjusted to pH 3 with 1 N HCl. The mixture was concentrated in vacuo. The crude product was purified by Prep-HPLC using a gradient of 0.1% TFA/ACN from 85:15 to 40:60 to give the titled product (10 mg, 24%) as a white solid. LC/MS: 394.9 [M+H]⁺.

1H NMR (400 MHz, MeOD) δ 4.47-4.31 (m, 1H), 4.30-4.13 (m, 2H), 4.06-3.78 (m, 3H), 3.74-3.53 (m, 4H), 3.17-3.06 (m, 1H), 2.98-2.67 (m, 3H), 2.54 (s, 3H), 2.45-2.33 (m, 2H), 2.31-2.16 (m, 1H), 2.14-1.96 (m, 5H), 1.95-1.76 (m, 2H), 1.73-1.54 (m, 2H).

### Example 2

### Preparation of 2-(1-methyl-10-oxo-4-(1-(pyridin-2-ylmethyl) azetidine-3-carbonyl)-1,4,9-triazaspiro [5.6]dodecan-9-yl) acetic acid (AC1069)

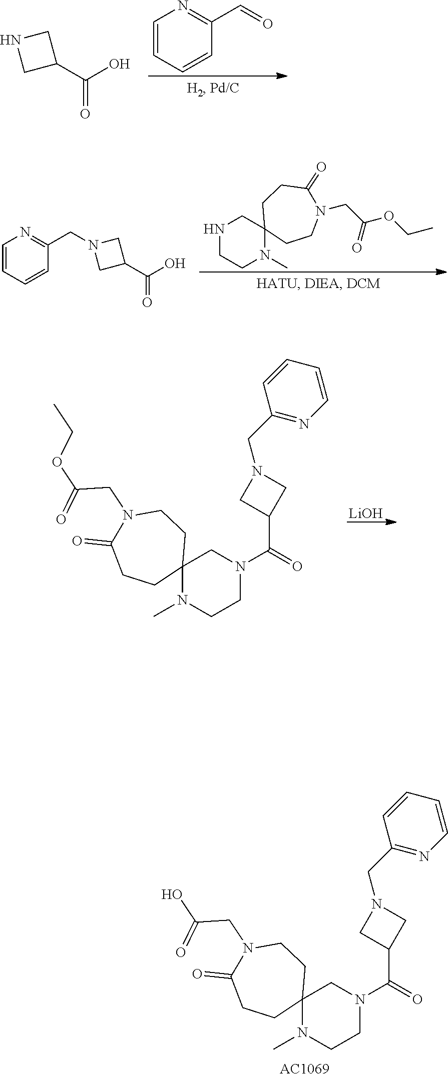

### Step 1: Preparation of 1-(pyridin-2-ylmethyl) azetidine-3-carboxylic acid

To a solution of azetidine-3-carboxylic acid (40 mg, 0.3734 mmol), pyridine-2-carbaldehyde (38 mg, 0.37 mmol) in MeOH (2 mL) was added Pd/C (40 mg, 40% in water). The reaction mixture was stirred at 25° C. for 2 h under H₂. The reaction mixture was filtered and concentrated to give the titled product (45 mg, 56.3%) as a white solid. LC/MS: 193.0 [M+H]⁺.

### Step 2: Preparation of ethyl 2-(1-methyl-10-oxo-4-(1-(pyridin-2-ylmethyl) azetidine-3-carbonyl)-1,4,9-triazaspiro [5.6]dodecan-9-yl) acetate

To a solution of 1-(pyridin-2-ylmethyl) azetidine-3-carboxylic acid (20 mg, 0.10 mmol), ethyl 2-{1-methyl-10-oxo-1,4,9-triazaspiro[5.6]dodecan-9-yl} acetate (29.5 mg, 0.10 mmol) and DIEA (40 mg, 0.3 mmol) in DCM (1 mL) was added HATU (59.4 mg, 0.15 mmol) at 25° C., the reaction mixture was stirred at 25° C. for 2 h. The reaction mixture was diluted with DCM (10 mL). The organic layers were washed with NaHCO₃, brine and dried over Na₂SO₄, then concentrated in vacuo to give the desired product (25 mg, 42%) as a yellow solid. LC/MS: 457.8 [M+H]⁺.

Step 3: Preparation of 2-(1-methyl-10-oxo-4-(1-(pyridin-2-ylmethyl) azetidine-3-carbonyl)-1,4,9-triazaspiro [5.6]dodecan-9-yl) acetic acid (AC1069)

To a solution of ethyl 2-(1-methyl-10-oxo-4-{[1-(pyridin-2-ylmethyl) azetidin-3-yl]carbonyl}-1,4,9-triazaspiro [5.6]dodecan-9-yl) acetate (25 mg, 0.06 mmol) in THF (0.6 mL) was added LiOH (5 mg, 0.12 mmol) in H₂O (0.2 mL) at 25° C., the reaction mixture was stirred at 25° C. for 2 h. The resulting solution was concentrated and purified by Prep-HPLC using a gradient of 0.1% FA/ACN from 80:20 to 40:60, and suitable fractions were pooled and lyophilized to give the desired product (10 mg, 43%) as a white solid.

LC/MS: 429.9[M+H]⁺; ¹H NMR (400 MHz, MeOD) δ 8.61-8.60 (m, 1H), 7.87-7.85 (m, 1H), 7.44-7.41 (m, 2H), 4.70-4.60 (m, 2H), 4.55-4.40 (m, 4H), 4.38-4.30 (m, 1H), 425-4.39 (m, 4H), 3.80-3.74 (m, 2H), 3.52-3.32 (m, 4H), 2.98-2.93 (m, 4H), 2.46-2.41 (m, 2H), 2.10-2.05 (m, 2H), 1.98-1.92 (m, 1H).

### Example 3

### Preparation of 2-(4-(cyclobutanecarbonyl)-1-methyl-10-oxo-1,4,9-triazaspiro[5.6]dodecan-9-yl)acetic acid (AC563)

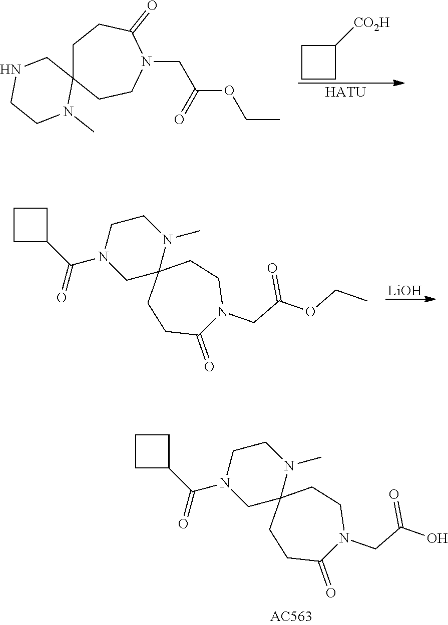

### Step 1: Preparation of ethyl 2-(4-(cyclobutanecarbonyl)-1-methyl-10-oxo-1,4,9-triazaspiro[5.6]dodecan-9-yl)acetate

To a solution of ethyl 2-(1-methyl-10-oxo-1,4,9-triazaspiro[5.6]dodecan-9-yl)acetate (150 mg, TFA salt, crude), cyclobutanecarboxylic acid (22 mg, 0.22 mmol) and HATU (125 mg, 0.33 mmol) in DMF (5 mL) was added DIEA (85 mg, 0.66 mmol). The mixture was stirred at room temperature overnight. The solvent was removed in vacuum and the residue was purified by chromatography with DCM/MeOH=0-20% to give the title compound (40 mg, 50% for two steps) as a white solid. LC/MS: 366.5 [M+H]⁺.

### Step 2: Preparation of 2-(4-(cyclobutanecarbonyl)-1-methyl-10-oxo-1,4,9-triazaspiro[5.6]dodecan-9-yl)acetic acid

To a solution of ethyl 2-(4-(cyclobutanecarbonyl)-1-methyl-10-oxo-1,4,9-triazaspiro[5.6]dodecan-9-yl)acetate (40 mg, 0.11 mmol) in THF-MeOH-water (2 mL, 3/1/1) was added LiOH (18 mg, 0.44 mmol). The mixture was stirred at 45° C. for 2 hours. The solvent was removed in vacuum and the residue was purified by Prep-HPLC (MeCN-water-TFA) to give the title compound (30 mg, 80.8%) as a white solid. LC/MS: 338.3 [M+H]⁺; ¹H NMR (400 MHz, MeOD) δ 4.39 (d, J=17.6 Hz, 1H), 4.12-3.58 (m, 4H), 3.57-3.33 (m, 6H), 3.05-2.84 (m, 4H), 2.55-1.77 (m, 11H).

### Examples 4 and 5

### Preparation of (R)-2-(4-(cyclobutanecarbonyl)-1-methyl-10-oxo-1,4,9-triazaspiro[5.6]dodecan-9-yl)acetic acid and (S)-2-(4-(cyclobutanecarbonyl)-1-methyl-10-oxo-1,4,9-triazaspiro[5.6]dodecan-9-yl)acetic acid (AC618 and AC619)

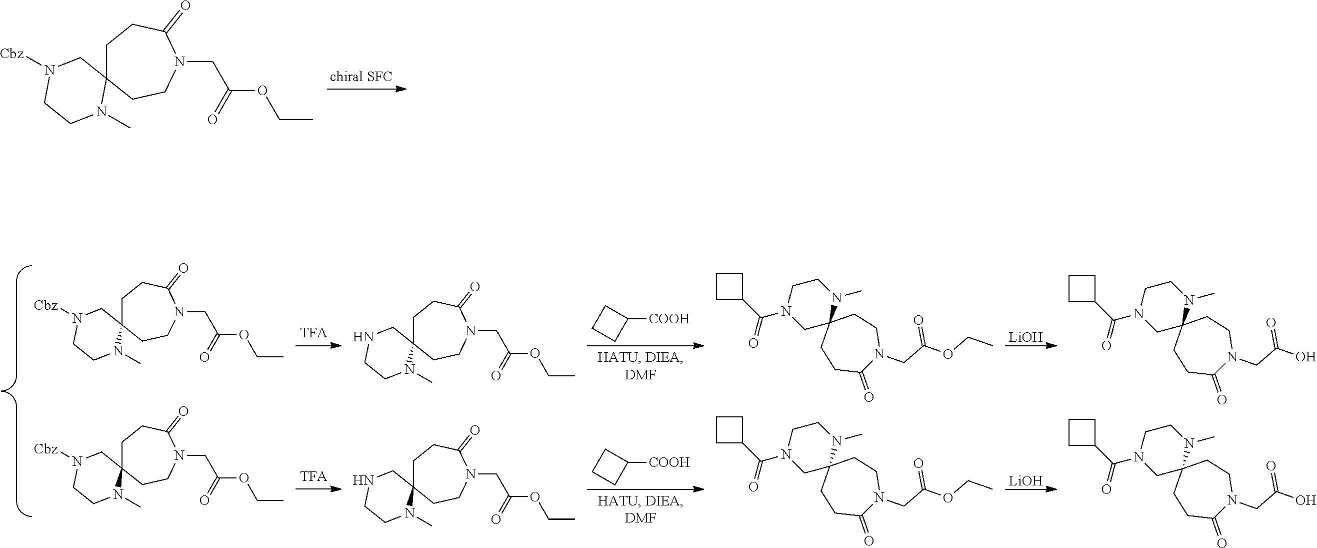

### Step 1: Preparation of benzyl (R)-9-(2-ethoxy-2-oxoethyl)-1-methyl-10-oxo-1,4,9-triazaspiro [5.6]dodecane-4-carboxylate and benzyl (S)-9-(2-ethoxy-2-oxoethyl)-1-methyl-10-oxo-1,4,9-tri azaspiro[5.6]dodecane-4-carboxylate

Benzyl 9-(2-ethoxy-2-oxoethyl)-1-methyl-10-oxo-1,4,9-triazaspiro[5.6]dodecane-4-carboxylate (550 mg, 1.37 mmol) was separated by SFC (Apparatus: Thar Prep 80; Column: CHIRALPAK IC (250 mm×20 mm, 5 μm); Modifier: 40% EtOH (NH₄OH 0.2%); Flow rate: 40 mL/min) to give two enantiomers. Peak 1: 250 mg as a white solid and Peak 2: 250 mg as a white solid. Each enantiomer was converted to the titled final products with the procedure described above.

Product obtained from the first peak: LC/MS: 338.3 [M+H]⁺; ¹H NMR (400 MHz, MeOD) δ 4.27-3.93 (m, 2H), 3.83-3.33 (m, 7H), 3.02-2.73 (m, 3H), 2.65-2.38 (m, 4H), 2.32-2.13 (m, 5H), 2.09-1.94 (m, 2H), 1.89-1.60 (m, 3H).

Product obtained from the second peak: LC/MS: 338.3 [M+H]⁺; ¹H NMR (400 MHz, MeOD) δ 4.27-3.93 (m, 2H), 3.83-3.33 (m, 7H), 3.02-2.73 (m, 3H), 2.65-2.38 (m, 4H), 2.32-2.13 (m, 5H), 2.11-1.80 (m, 3H), 1.79-1.55 (m, 2H).

### Example 6

### Preparation of 2-(9-oxo-2-(2-phenylpropanoyl)-7-oxa-2,10-diazaspiro[5.6]dodecan-10-yl)acetic acid (AC890)

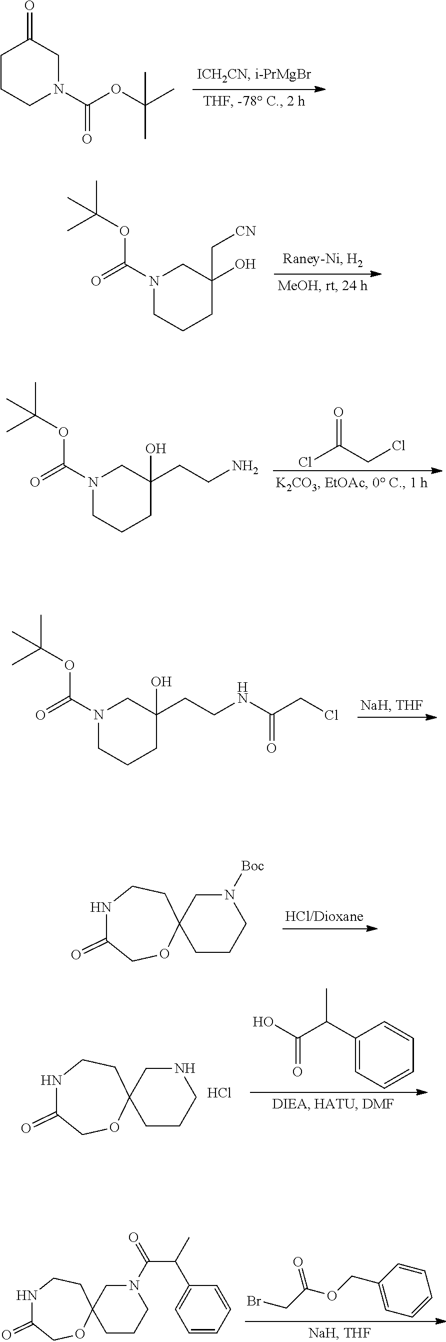

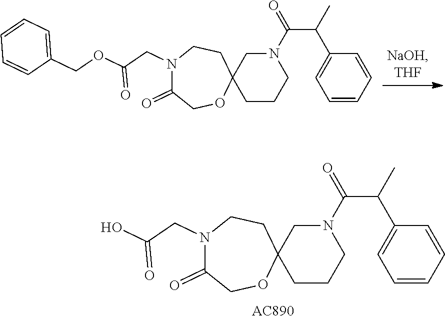 [SMILES: CC(C(=O)N1CCCC2(CCN(CC(=O)OCc3ccccc3)C(=O)CO2)C1)c1ccccc1 ; CC(C(=O)N1CCCC2(CCN(CC(=O)O)C(=O)CO2)C1)c1ccccc1]

### Step 1: Preparation of tert-butyl 3-(cyanomethyl)-3-hydroxypiperidine-1-carboxylate

To a mixture of tert-butyl 3-oxopiperidine-1-carboxylate (10 g, 50.2 mmol) and 2-iodoacetonitrile (8.38 g, 50.2 mmol) in THF (250 mL) was added i-PrMgBr (52.7 mL, 52.7 mmol, 1M) slowly at −78° C. under nitrogen. The reaction was stirred at −78° C. for 2 hr. The reaction was quenched with water, NaHCO₃ aqueous solution and extracted with EA (150 mL×3). The organic layer was washed with brine and dried over Na₂SO₄, filtered and the filtrate was concentrated. The crude product was purified by silica gel column chromatography using 20%˜30% EtOAc in PE as eluent to afford the desired compound (7.0 g, 52.8%). ¹HNMR (400 MHz, CDCl₃): δ 3.82-3.56 (m, 2H), 3.35-3.21 (m, 2H), 2.56 (s, 2H), 1.84-1.68 (m, 3H), 1.59-1.40 (m, 10H).

### Step 2: Preparation of tert-butyl 3-(2-aminoethyl)-3-hydroxypiperidine-1-carboxylate

To a mixture of tert-butyl 3-(cyanomethyl)-3-hydroxypiperidine-1-carboxylate (5.0 g, 20.8 mmol) in MeOH (200 mL) was added Raney Nickel (3.56 g, 41.6 mmol) portion wise. The reaction was stirred at RT for 24 h. The mixture was filtered and the filtrate was concentrated to afford the desired compound (4.3 g crude) which was used in next step without further purification.

### Step 3: Preparation of tert-butyl 3-(2-(2-chloroacetamido)ethyl)-3-hydroxypiperidine-1-carboxylate

2-chloroacetyl chloride (2.31 g, 20.4 mmol) was added dropwise to a vigorously stirred mixture of tert-butyl 3-(2-aminoethyl)-3-hydroxypiperidine-1-carboxylate (4.3 g, 20.4 mmol) in EA (150 mL) and potassium carbonate aqueous solution (5.65 g, 40.9 mmol dissolved in 100 mL water) at 0° C. The mixture was stirred at 0° C. for 2 h and extracted with ethyl acetate. The organic layer was dried over sodium sulphate, filtered and evaporated under reduced pressure. The crude residue was purified by silica gel column chromatography using EtOAc as eluent to afford the desired compound (3.8 g, 56.9%). LC/MS: 342.9 [M+Na]⁺.

### Step 4: Preparation of tert-butyl 9-oxo-7-oxa-2,10-diazaspiro[5.6]dodecane-2-carboxylate

To a mixture of tert-butyl 3-[2-(2-chloroacetamido)ethyl]-3-hydroxypiperidine-1-carboxylate (3.8 g, 11.5 mmol) in THF (1 L) was added NaH (748 mg, 18.7 mmol, 60%) portion wise at 0° C. The reaction was stirred at 70° C. for 2 hr, quenched with sat. NH₄Cl solution and extracted with EA (500 mL×3). The organic layer was washed with brine and dried over Na₂SO₄, filtered and the filtrate was concentrated. The crude residue was purified by silica gel column chromatography using 100% EA as eluent to afford the desired compound (1.3 g, 33%). ¹H NMR (300 MHz, CDCl3) δ 6.52-6.50 (m, 1H), 4.35-4.15 (m, 2H), 3.61-3.06 (m, 6H), 2.11-1.81 (m, 2H), 1.73-1.65 (m, 4H), 1.52 (s, 9H).

### Step 5: Preparation of 7-oxa-2,10-diazaspiro[5.6]dodecan-9-one

To a mixture of tert-butyl {9-oxo-7-oxa-2,10-diazaspiro[5.6]dodecan-2-yl}formate (1.3 g, 4.56 mmol) in DCM (4 mL) was added HCl (4 mL, 4M in 1,4-dioxane) slowly. The reaction was stirred at 25° C. for 4 hr. The reaction mixture was concentrated under vacuum to afford the desired product (1.1 g, 98.5%) which was used in next step without further purification. LC/MS: 185.1 [M+H]⁺.

### Step 6: Preparation of 2-(2-phenylpropanoyl)-7-oxa-2,10-diazaspiro[5.6]dodecan-9-one

To a mixture of 2-phenylpropanoic acid (75 mg, 0.50 mmol) in DMF (3 mL) was added HATU (190 mg, 0.50 mmol) portion wise at 20° C. The solution was stirred at 25° C. for 30 min. A solution of 7-oxa-2,10-diazaspiro[5.6]dodecan-9-one (154.3 mg, 0.7 mmol) and DIEA (323 mg 2.5 mmol) in DMF (2 mL) was added and stirred at 20° C. for 16 hr. The reaction was diluted with EA (50 mL). The organic layer was washed with brine and dried over Na₂SO₄, filtered and the filtrate was concentrated. The crude residue was purified by silica gel column chromatography using 15% MeOH in DCM as eluent to afford the desired product (90 mg, 51.3%). LC/MS: 317.1 [M+H]⁺.

Step 7: Preparation of benzyl 2-(9-oxo-2-(2-phenylpropanoyl)-7-oxa-2,10-diazaspiro [5.6]dodecan-10-yl)acetate

To a solution of 2-(2-phenylpropanoyl)-7-oxa-2,10-diazaspiro[5.6]dodecan-9-one (90 mg, 0.28 mmol) in THF (4 mL) was added NaH (56.9 mg, 1.42 mmol, 60%) portion wise. The reaction was stirred at 25° C. for 0.5 h. Then a solution of benzyl 2-bromoacetate (325.9 mg 1.42 mmol) in THF (1 mL) was added at 25° C. under nitrogen. The reaction was stirred at 25° C. for 48 hr. The reaction was quenched with sat. NH₄Cl solution and extracted with EA (10 mL×3). The organic layer was washed with brine and dried over Na₂SO₄, filtered and the filtrate was concentrated. The crude product was purified by silica gel column chromatography using 15% MeOH in DCM as eluent to afford the desired product (65 mg, 31.9%). LC/MS: 464.9 [M+H]⁺.

### Step 8: Preparation of 2-(9-oxo-2-(2-phenylpropanoyl)-7-oxa-2,10-diazaspiro[5.6]dodecan-10-yl)acetic acid (AC890)

To a solution of benzyl 2-[9-oxo-2-(2-phenylpropanoyl)-7-oxa-2,10-diazaspiro[5.6]dodecan-10-yl]acetate (65 mg, 0.091 mmol) in THF (3 mL) was added NaOH (18 mg, 0.45 mmol) in H₂O (1 mL). After stirring at 25° C. for 4 hr, the reaction was quenched with H₂O and extracted with Et₂O (10 mL×3). The aqueous layer was purified by Prep-HPLC using a gradient of 0.1% TFA/ACN from 75:25 to 45:55 to give the titled product (24.4 mg, 66.8%). LC/MS: 375.0 [M+H]⁺; ¹H NMR (400 MHz, CD₃OD) δ 7.47-7.13 (m, 5H), 4.59-4.56 (m, 0.5H), 4.39-4.26 (m, 1H), 4.24-4.01 (m, 4H), 3.88-3.75 (m, 1H), 3.64-3.35 (m, 2.5H), 3.25-3.02 (m, 1H), 2.95-2.88 (m, 0.5H), 2.76-2.72 (m, 0.5H), 2.13-1.78 (m, 3H), 1.77-1.40 (m, 2H), 1.39-1.35 (m, 3H), 1.16-0.89 (m, 1H).

### Example 7

### Preparation of 2-(9-oxo-2-((R)-2-phenylpropanoyl)-7-oxa-2,10-diazaspiro[5.6]dodecan-10-yl)acetic acid (AC1028)

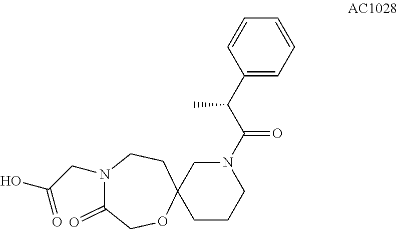 [SMILES: C[C@@H](C(=O)N1CCCC2(CCN(CC(=O)O)C(=O)CO2)C1)c1ccccc1]

2-(9-Oxo-2-((R)-2-phenylpropanoyl)-7-oxa-2,10-diazaspiro[5.6]dodecan-10-yl)acetic acid was prepared analogously with the procedure described for 2-(9-oxo-2-(2-phenylpropanoyl)-7-oxa-2,10-diazaspiro[5.6]dodecan-10-yl)acetic acid. LC/MS: 374.9 [M+H]⁺; ¹H NMR (400 MHz, CD₃OD) δ 7.36-7.16 (m, 5H), 4.60-4.55 (m, 0.5H), 4.21-4.11 (m, 1H), 4.09-3.98 (m, 4H), 3.88-3.82 (m, 1H), 3.52-3.46 (m, 2H), 3.38-3.31 (m, 0.5H), 3.25-3.03 (m, 1H), 2.94-2.88 (m, 0.5H), 2.76-2.72 (m, 0.5H), 1.97-1.72 (m, 3H), 1.66-1.44 (m, 2H), 1.40-1.28 (m, 3H), 1.12-0.88 (m, 1H).

### Examples 8 and 9

### Preparation of 2-((S)-9-oxo-2-((S)-2-phenylpropanoyl)-7-oxa-2,10-diazaspiro[5.6]dodecan-10-yl)acetic acid and 2-((R)-9-oxo-2-((S)-2-phenylpropanoyl)-7-oxa-2,10-diazaspiro[5.6]dodecan-10-yl)acetic acid (AC978 and AC979)

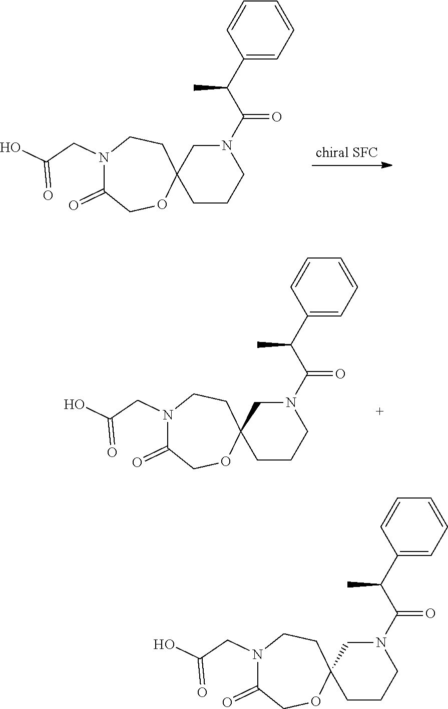 [SMILES: C[C@H](C(=O)N1CCCC2(CCN(CC(=O)O)C(=O)CO2)C1)c1ccccc1 ; C[C@H](C(=O)N1CCC[C@]2(CCN(CC(=O)O)C(=O)CO2)C1)c1ccccc1 ; C[C@H](C(=O)N1CCC[C@@]2(CCN(CC(=O)O)C(=O)CO2)C1)c1ccccc1]

The racemate compound of 2-(9-oxo-2-((S)-2-phenylpropanoyl)-7-oxa-2,10-diazaspiro[5.6]dodecan-10-yl)acetic acid was prepared with the same procedure as described for 2-(9-oxo-2-(2-phenylpropanoyl)-7-oxa-2,10-diazaspiro[5.6]dodecan-10-yl)acetic acid, and was separated by chiral SFC (Column: CHIRALPAK AD-H 250 mm×20 mm, 5 μm; Modifier: CO₂ and 40% IPA (0.2% NH₄OH); Flow rate: 40 mL/min) to give two diastereomers of 2-((S)-9-oxo-2-((S)-2-phenylpropanoyl)-7-oxa-2,10-diazaspiro[5.6]dodecan-10-yl)acetic acid (19.8 mg, 35%) and 2-((R)-9-oxo-2-((S)-2-phenylpropanoyl)-7-oxa-2,10-diazaspiro[5.6]dodecan-10-yl)acetic acid (22.6 mg, 40%). LC/MS: 374.8 [M+H]⁺. ¹H NMR of first peak diastereomer: (400 MHz, CD₃OD): δ 7.33-7.20 (m, 5H), 4.59-4.56 (m, 0.5H), 4.36-4.34 (m, 1H), 4.18-4.15 (m, 1H), 4.10-4.05 (m, 3H), 3.82-3.81 (m, 1H), 3.53-3.47 (m, 2H), 3.18-3.03 (m, 1.5H), 2.91-2.81 (m, 0.5H), 2.75-2.72 (m, 0.5H), 2.15-2.07 (m, 1H), 1.98-1.85 (m, 1H), 1.81-1.77 (m, 1H), 1.75-1.54 (m, 2H), 1.42-1.32 (m, 3H), 0.97-0.91 (m, 1H); ¹H NMR of second peak diastereomer: (400 MHz, CD₃OD): δ 7.33-7.16 (m, 5H), 4.42-4.39 (m, 1H), 4.12-4.05 (m, 4H), 3.99-3.78 (m, 1H), 3.57-3.46 (m, 3H), 3.25-3.09 (m, 1H), 2.90-2.88 (m, 0.5H), 2.72-2.65 (m, 0.5H), 2.15-1.91 (m, 1H), 1.89-1.81 (m, 2H), 1.62-1.46 (m, 2H), 1.38-1.35 (m, 3H), 1.08-1.04 (m, 1H).

### Example 10

### Preparation of 2-(4-(6-acetyl-5,6,7,8-tetrahydropyrido[4,3-d]pyrimidin-2-yl)-1-methyl-10-oxo-1,4,9-triazaspiro[5.6]dodecan-9-yl)acetic acid (AC1030)

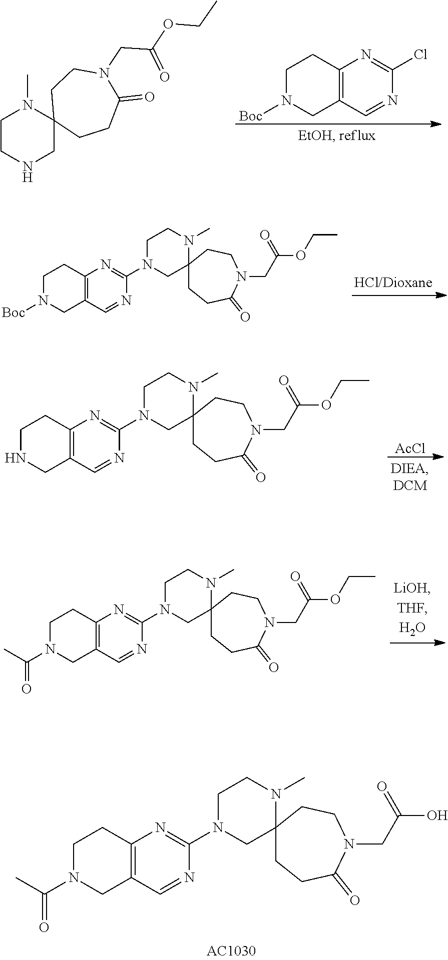

### Step 1: Preparation of tert-butyl 2-(9-(2-ethoxy-2-oxoethyl)-1-methyl-10-oxo-1,4,9-triazaspiro [5.6]dodecan-4-yl)-7,8-dihydropyrido[4,3-d]pyrimidine-6(5H)-carboxylate

To a solution of ethyl 2-(1-methyl-10-oxo-1,4,9-triazaspiro[5.6]dodecan-9-yl)acetate (80 mg, 0.28 mmol) in EtOH (5 mL) was added tert-butyl 2-chloro-7,8-dihydropyrido[4,3-d]pyrimidine-6(5H)-carboxylate (76 mg, 0.28 mmol). The reaction was stirred at 80° C. for 48 h. The reaction mixture was concentrated in vacuum and purified by Combi-flash (DCM/MeOH=10:1) to afford product (60 mg, 41%) as a white solid. LC/MS: 517[M+H]⁺.

### Step 2: Preparation of ethyl 2-(1-methyl-10-oxo-4-(5,6,7,8-tetrahydropyrido[4,3-d]pyrimidin-2-yl)-1,4,9-triazaspiro[5.6]dodecan-9-yl)acetate

A solution of tert-butyl 2-(9-(2-ethoxy-2-oxoethyl)-1-methyl-10-oxo-1,4,9-triazaspiro[5.6]dodecan-4-yl)-7,8-dihydropyrido[4,3-d]pyrimidine-6(5H)-carboxylate (60 mg, 0.12 mmol) in HCl/Dioxane (5 mL) was stirred at 25° C. for 1 h. The reaction mixture was concentrated in vacuo to afford product (50 mg, crude) as a light yellow solid. LC/MS: 417[M+H]⁺.

### Step 3: Preparation of ethyl 2-(4-(6-acetyl-5,6,7,8-tetrahydropyrido[4,3-d]pyrimidin-2-yl)-1-methyl-10-oxo-1,4,9-triazaspiro[5.6]dodecan-9-yl)acetate

To a solution of ethyl 2-(1-methyl-10-oxo-4-(5,6,7,8-tetrahydropyrido[4,3-d]pyrimidin-2-yl)-1,4,9-triazaspiro[5.6]dodecan-9-yl)acetate (50 mg, 0.12 mmol) in DCM (8 mL) was added DIEA (123 mg, 0.95 mmol) and acetyl chloride (15 mg, 0.12 mmol). The reaction mixture was stirred at 25° C. for 1 h. The reaction mixture was washed with brine. The organic layer was dried over Na₂SO₄ and concentrated in vacuo to afford product (70 mg, crude) as a light yellow solid. LC/MS: 459 [M+H]⁺.

### Step 4: Preparation of 2-(4-(6-acetyl-5,6,7,8-tetrahydropyrido[4,3-d]pyrimidin-2-yl)-1-methyl-10-oxo-1,4,9-triazaspiro[5.6]dodecan-9-yl)acetic acid

To a solution of ethyl 2-(4-(6-acetyl-5,6,7,8-tetrahydropyrido[4,3-d]pyrimidin-2-yl)-1-methyl-10-oxo-1,4,9-triazaspiro[5.6]dodecan-9-yl)acetate (70 mg, 0.12 mmol) in THF (6 mL) was added a solution of LiOH. H₂O (44 mg, 1.05 mmol) in H₂O (2 mL). The reaction mixture was stirred at 25° C. for 1 h. The reaction mixture was concentrated in vacuo. The crude residue was purified by Prep-HPLC to afford product (20.3 mg, 30.9%) as a white solid. LC/MS: 431 [M+H]⁺.

¹H NMR (400 MHz, MeOD) δ 8.24 (d, J=4.8 Hz, 1H), 5.41-5.37 (m, 1H), 4.86-4.84 (m, 1H), 4.62-4.58 (m, 2H), 4.36-4.32 (m, 1H), 4.04-3.99 (m, 2H), 3.88-3.77 (m, 2H), 3.53-3.36 (m, 4H), 3.25-3.21 (m, 1H), 3.07-3.04 (m, 1H), 2.97-2.85 (m, 4H), 2.79 (t, J=6.0 Hz, 1H), 2.67-2.34 (m, 2H), 2.25-1.77 (m, 6H).

### Example 11

### Preparation of 2-(4-(2-amino-5-chloro-6-methylpyrimidin-4-yl)-1-methyl-10-oxo-1,4,9-triazaspiro [5.6]dodecan-9-yl) acetic acid (AC1031)

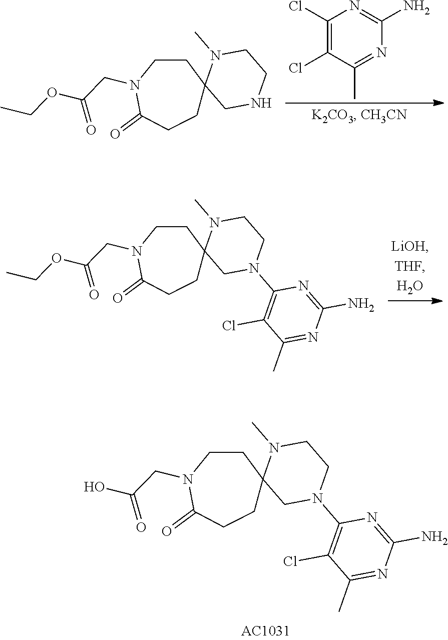

### Step 1: Preparation of ethyl 2-(4-(2-amino-5-chloro-6-methylpyrimidin-4-yl)-1-methyl-10-oxo-1,4,9-triazaspiro[5.6]dodecan-9-yl)acetate

To a solution of ethyl 2-(1-methyl-10-oxo-1,4,9-triazaspiro[5.6]dodecan-9-yl)acetate (30 mg, 0.11 mmol) in CH₃CN (5 mL) was added 4,5-dichloro-6-methylpyrimidin-2-amine (19 mg, 0.11 mmol) and K₂CO₃ (29 mg, 0.22 mmol). The reaction mixture was stirred at 80° C. for 16 h. The mixture was filtered and the filtrate was concentrated in vacuo to afford product (60 mg, crude) as a light yellow solid which was used without further purification. LC/MS: 425 [M+H]⁺.

### Step 2: Preparation of 2-(4-(2-amino-5-chloro-6-methylpyrimidin-4-yl)-1-methyl-10-oxo-1,4,9-triazaspiro[5.6]dodecan-9-yl)acetic acid

To a solution of ethyl 2-(4-(2-amino-5-chloro-6-methylpyrimidin-4-yl)-1-methyl-10-oxo-1,4,9-triazaspiro[5.6]dodecan-9-yl)acetate (60 mg, crude) in THF (6 mL) was added a solution of LiOH·H₂O (40 mg, 0.95 mmol) in H₂O (2 mL). The reaction mixture was stirred at 25° C. for 1 h. The reaction mixture was concentrated in vacuo and the residue was purified by Prep-HPLC to afford the titled product (10.4 mg, 17.8%) as a white solid. LC/MS: 397 [M+H]⁺.

¹H NMR (400 MHz, MeOD) δ 4.66-4.14 (m, 4H), 4.10-3.99 (m, 2H), 3.53-3.43 (m, 2H), 3.36-3.35 (m, 1H), 3.13-2.98 (m, 1H), 2.92 (s, 3H), 2.51-2.33 (m, 5H), 2.16-2.07 (m, 2H), 2.01-2.00 (m, 1H).

### Example 12

### Representative KLHDC2 Binding Assay

The following is a description of an assay for evaluating ligand binding to KLHDC2.

Amplified Luminescence Proximity Homogenous Assay (AlphaScreen) was used to monitor protein-protein interaction of two tagged components that are immobilized on beads. One component was GST-KLHDC2 (E3) and the other one was biotinylated-SelK peptide with a length of 12 amino acids (Substrate peptide). When the two components bind, they bring the Alpha beads in close proximity to each other that results in a luminescent signal. A compound that has affinity for KLHDC2 will compete with the biotinylated-SelK peptide, preventing the beads from being in proximity to each other, therefore, reducing the luminescent signal in a dose dependent manner. The effects of DMSO on the AlphaScreen readout were tested and it was established that this organic solvent used to dissolve most of the hit compounds has detectable but marginal effects on the assay. When DMSO was kept below 5%, its effect on the AlphaScreen assay was negligible.

AlphaScreen assays for determining and measuring protein-protein interactions were performed using EnSpire reader (PerkinElmer). GST-tagged KLHDC2 was attached to anti-GST AlphaScreen acceptor beads. Synthetic biotinylated 12 aa SelK degron peptide (Bio-Synthesis, Inc.) was immobilized to streptavidin-coated AlphaScreen donor beads. The donor and acceptor beads were brought into proximity by the interactions between the SelK peptide and KLHDC2. Excitation of the donor beads by a laser beam of 680 nm promotes the formation of singlet oxygen. When an acceptor bead is in close proximity, the singlet oxygen reacts with thioxene derivatives in the acceptor beads and causes the emission of 520-620 nm photons, which are detected as the binding signal. If the beads are not in close proximity to each other, the oxygen will return to its ground state and the acceptor beads will not emit light. Competition assays were performed in the presence of representative binding compounds, which were titrated at various concentrations.

The experiments were conducted with 0.12 nM of GST-KLHDC2 and 1.7 nM biotinylated 12 aa SelK peptide in the presence of 5 μg/ml donor and acceptor beads in a buffer of 25 mM HEPES, pH 7.5, 100 mM NaCl, 1 mM TCEP, 0.1% Tween-20, and 0.05 mg/ml Bovine Serum Albumin. The concentrations of the compounds used in competition assays ranged from 0.1 nM to 25 mM. The experiments were done in triplicate. IC₅₀ values were determined using non-linear curve fitting of the dose response curves generated with Prism 4 (GraphPad).

Results for representative binding compounds of formulae (II)-(IV) are summarized in Tables 2, 4, and 6, respectively.

While illustrative embodiments have been illustrated and described, it will be appreciated that various changes can be made therein without departing from the spirit and scope of the invention.

1. A compound having formula (I): 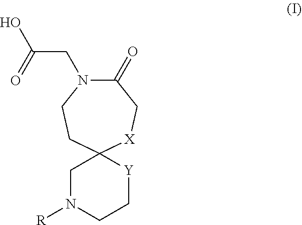
or a pharmaceutically acceptable salt or ester thereof,
wherein
X is CH₂ or O;
Y is NCH₃ or CH₂; and
R is alkylcarbonyl, arylcarbonyl, cycloalkylcarbonyl, alkoxycarbonyl, aryloxycarbonyl, heterocyclic alkylcarbonyl, heteroarylcarbonyl, alkylaminocarbonyl, arylaminocarbonyl, alkyl, aryl, or heteroaryl.

2. A compound having formula (II): 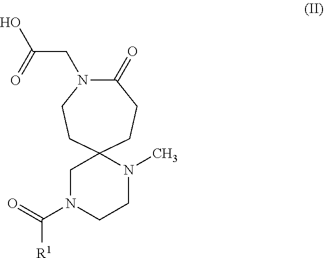
or a pharmaceutically acceptable salt or ester thereof,
wherein R¹ is alkyl, aryl, cycloalkyl, alkoxy, aryloxy, heterocyclic alkyl, heteroaryl, alkylamino, or arylamino.

3. The compound of claim 2, wherein the compound is selected from the group consisting of 2-(1-methyl-10-oxo-4-(2-phenylcyclopropane-1-carbonyl)-1,4,9-triazaspiro[5.6]dodecan-9-yl)acetic acid, 2-(1-methyl-4-(2-methyl-3-phenylpropanoyl)-10-oxo-1,4,9-triazaspiro[5.6]dodecan-9-yl)acetic acid, 2-(4-(3,3-difluorocyclobutane-1-carbonyl)-1-methyl-10-oxo-1,4,9-triazaspiro[5.6]dodecan-9-yl)acetic acid, 2-(4-(1-(4-fluorophenyl)cyclopropane-1-carbonyl)-1-methyl-10-oxo-1,4,9-triazaspiro[5.6]dodecan-9-yl)acetic acid, 2-(1-methyl-4-(2-methyl-2-phenoxypropanoyl)-10-oxo-1,4,9-triazaspiro[5.6]dodecan-9-yl)acetic acid, 2-(4-((1,3-trans)-3-((tert-butoxycarbonyl)amino)cyclobutane-1-carbonyl)-1-methyl-10-oxo-1,4,9-triazaspiro[5.6]dodecan-9-yl)acetic acid, 2-(4-(1-hydroxycyclopentane-1-carbonyl)-1-methyl-10-oxo-1,4,9-triazaspiro[5.6]dodecan-9-yl)acetic acid, 2-(4-(cyclobutylcarbamoyl)-1-methyl-10-oxo-1,4,9-triazaspiro[5.6]dodecan-9-yl)acetic acid, 2-(1-methyl-10-oxo-4-(phenylcarbamoyl)-1,4,9-triazaspiro[5.6]dodecan-9-yl)acetic acid, 2-(4-(benzylcarbamoyl)-1-methyl-10-oxo-1,4,9-triazaspiro[5.6]dodecan-9-yl)acetic acid, 2-(4-(cyclopropanecarbonyl)-1-methyl-10-oxo-1,4,9-triazaspiro[5.6]dodecan-9-yl)acetic acid, 2-(4-(2-(4-hydroxyphenyl)propanoyl)-1-methyl-10-oxo-1,4,9-triazaspiro[5.6]dodecan-9-yl)acetic acid, 2-(4-(2-methoxy-2-phenylpropanoyl)-1-methyl-10-oxo-1,4,9-triazaspiro[5.6]dodecan-9-yl)acetic acid, 2-(1-methyl-10-oxo-4-(1-phenyl-1H-pyrazole-4-carbonyl)-1,4,9-triazaspiro[5.6]dodecan-9-yl)acetic acid, 2-(4-((S)-1-(tert-butoxycarbonyl)azetidine-2-carbonyl)-1-methyl-10-oxo-1,4,9-triazaspiro[5.6]dodecan-9-yl)acetic acid, 2-(4-(1-benzyl-5-oxopyrrolidine-3-carbonyl)-1-methyl-10-oxo-1,4,9-triazaspiro[5.6]dodecan-9-yl)acetic acid, 2-(4-(1-acetylpiperidine-4-carbonyl)-1-methyl-10-oxo-1,4,9-triazaspiro[5.6]dodecan-9-yl)acetic acid, 2-(1-methyl-10-oxo-4-(1-(pyridin-2-ylmethyl)azetidine-3-carbonyl)-1,4,9-triazaspiro[5.6]dodecan-9-yl)acetic acid, 2-(4-(1-benzoylazetidine-3-carbonyl)-1-methyl-10-oxo-1,4,9-triazaspiro[5.6]dodecan-9-yl)acetic acid, 2-(4-(benzoyl-D-prolyl)-1-methyl-10-oxo-1,4,9-triazaspiro[5.6]dodecan-9-yl)acetic acid, and 2-(4-(acetyl-D-prolyl)-1-methyl-10-oxo-1,4,9-triazaspiro[5.6]dodecan-9-yl)acetic acid, or a pharmaceutically acceptable salt or ester thereof.

4. A compound having formula (III): 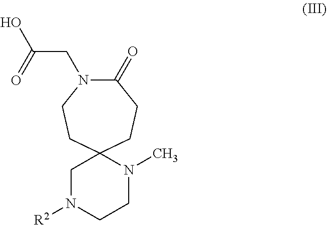
or a pharmaceutically acceptable salt or ester thereof,
wherein R² is alkyl, heteroatom substituted alkyl, cycloalkyl, bicyclic alkyl, aryl, heteroaryl, bicyclic aryl, or heterobicyclic aryl.

5. The compound of claim 4, wherein the compound is selected from the group consisting of 2-(4-(6-acetyl-5,6,7,8-tetrahydropyrido[4,3-d]pyrimidin-2-yl)-1-methyl-10-oxo-1,4,9-triazaspiro[5.6]dodecan-9-yl)acetic acid, 2-(4-(2-amino-5-chloro-6-methylpyrimidin-4-yl)-1-methyl-10-oxo-1,4,9-triazaspiro[5.6]dodecan-9-yl)acetic acid, and 2-(9-(carboxymethyl)-1-methyl-10-oxo-1,4,9-triazaspiro[5.6]dodecan-4-yl)pyrimidine-4-carboxylic acid, or a pharmaceutically acceptable salt or ester thereof.

6. A compound having formula (IV): 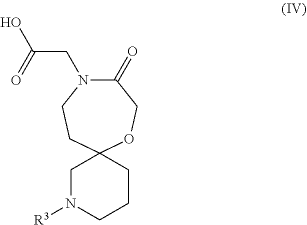
or a pharmaceutically acceptable salt or ester thereof,
wherein R³ is alkylcarbonyl, arylcarbonyl, cycloalkylcarbonyl, alkoxycarbonyl, aryloxycarbonyl, heterocyclic alkylcarbonyl, heteroarylcarbonyl, or alkylaminocarbonyl, arylaminocarbonyl.

7. The compound of claim 6, wherein the compound is selected from the group consisting of 2-(9-oxo-2-(2-phenylpropanoyl)-7-oxa-2,10-diazaspiro[5.6]dodecan-10-yl)acetic acid, 2-((S)-9-oxo-2-((S)-2-phenylpropanoyl)-7-oxa-2,10-diazaspiro[5.6]dodecan-10-yl)acetic acid, 2-((R)-9-oxo-2-((S)-2-phenylpropanoyl)-7-oxa-2,10-diazaspiro[5.6]dodecan-10-yl)acetic acid, 2-(9-oxo-2-((R)-2-phenylpropanoyl)-7-oxa-2,10-diazaspiro[5.6]dodecan-10-yl)acetic acid, 2-(2-(cyclopentanecarbonyl)-9-oxo-7-oxa-2,10-diazaspiro[5.6]dodecan-10-yl)acetic acid, 2-(2-(cyclobutanecarbonyl)-9-oxo-7-oxa-2,10-diazaspiro[5.6]dodecan-10-yl)acetic acid, 2-(2-(2-(3-hydroxypyrrolidin-1-yl)pyrimidin-4-yl)-9-oxo-7-oxa-2,10-diazaspiro[5.6]dodecan-10-yl)acetic acid, 2-(2-(2-methylpyrimidin-4-yl)-9-oxo-7-oxa-2,10-diazaspiro[5.6]dodecan-10-yl)acetic acid, and 2-(9-oxo-2-(3-phenylpropanoyl)-7-oxa-2,10-diazaspiro[5.6]dodecan-10-yl)acetic acid, or a pharmaceutically acceptable salt or ester thereof.

8. A method for inhibiting the enzymatic activity of KLHDC2, comprising contacting KLHDC2 with an amount of a compound of claim 1, or a pharmaceutically acceptable salt or ester thereof, effective to inhibit KLHDC2 activity.

9. A method for inhibiting the enzymatic activity of KLHDC2, comprising contacting KLHDC2 with an amount of a compound of claim 2, or a pharmaceutically acceptable salt or ester thereof, effective to inhibit KLHDC2 activity.

10. A method for inhibiting the enzymatic activity of KLHDC2, comprising contacting KLHDC2 with an amount of a compound of claim 4, or a pharmaceutically acceptable salt or ester thereof, effective to inhibit KLHDC2 activity.

11. A method for inhibiting the enzymatic activity of KLHDC2, comprising contacting KLHDC2 with an amount of a compound of claim 6, or a pharmaceutically acceptable salt or ester thereof, effective to inhibit KLHDC2 activity.
